(19) United States
(12) Patent Application Publication
(10) Pub. No.: US 2018/0094200 A1
(43) Pub. Date: Apr. 5, 2018

(54) Hydrophobic Nucleic Acid Salts As Security Markers

(71) Applicant: APDN (B.V.I.) Inc., Road Town, Tortola (VG)

(72) Inventor: Maciej B. Szczepanik, Mount Sinai, NY (US)

(21) Appl. No.: 15/562,495

(22) PCT Filed: Apr. 20, 2016
(86) PCT No.: PCT/US16/28335

§ 371 Date: Sep. 28, 2017

Related U.S. Application Data
(60) Provisional application No. 62/150,092, filed on Apr. 20, 2015.

(51) Int. Cl. C10L 1/00; C12Q 1/68; C10L 1/10; G01N 33/28; G01N 33/44; G01N 33/02

(52) U.S. Cl. CPC C10L 1/003; C12Q 1/68; C10L 1/10; C10L 2270/026; G01N 33/44; G01N 33/02; G01N 33/28

(57) ABSTRACT

The invention provides a method of marking a hydrophobic medium with a nucleic acid marker, the method includes: providing a trialkylammonium salt of the nucleic acid marker, a tetraalkylphosphonium salt of the nucleic acid marker or a tetraarylphosphonium salt of the nucleic acid marker; and incorporating the trialkylammonium salt of the nucleic acid marker, the tetraalkylphosphonium salt of the nucleic acid marker or the tetraarylphosphonium salt of the nucleic acid marker into the hydrophobic medium. The hydrophobic medium can be authenticated after shipping or recovery from the stream of commerce by detecting the nucleic acid marker in a sample of the hydrophobic medium.

Nucleic acids, including DNA and RNA, provide an enormous number of potential discreet sequences and combinations of sequences, and therefore constitute exceptionally powerful information sources that may be used in a wide variety of applications, including for example, authenticity markers, security markers, or tracking and tracing tags. One major disadvantage of nucleic acids as markers, however, is their insolubility in a wide variety of non-aqueous media, such as hydrophobic and organic solvent-based media. Although DNA can be delivered and fairly evenly distributed as an aqueous solution in relatively hydrophobic media with assistance of specific excipients or perturbants (Liang et al. US Pat. Appl. 2014/0099643), certain applications and carriers do not tolerate even miniscule amounts of water.

Hydrophobic, lipid soluble forms of DNA, have been used in the fields of medicine, and basic research in biochemistry, genetics and molecular biology. For these applications, DNA is typically rendered hydrophobic by encapsulation by or association with lipids (Wheeler et al. U.S. Pat. No. 7,422,902; Zhang et al. U.S. Pat. No. 6,110,745) or in lipid and phospholipid emulsions for the purpose of targeted gene therapy, drug stabilization and drug delivery (Huang et U.S. Pat. No. 6,890,557).

### SUMMARY

The present invention provides a novel, efficient method to convert a water-soluble nucleic acid salts into hydrophobic forms that are soluble in organic solvents. The method allows delivery of hydrophobic nucleic acids into the hydrophobic and organic solvent carrier in the absence of water or a partially aqueous intermediary, and facilitates generally homogenous distribution of the hydrophobic nucleic acids. These hydrophobic nucleic acids may be used for a wide variety of purposes and applications that may provide information associated with a specific hydrophobic nucleic acid. A specific hydrophobic nucleic acid may be associated with, for example, an identified lot or batch of a product; an expiry date of goods, security information, material tracking information, transport information, covert information, path to market or path to manufacturer information, and the like. Any of these suggested uses may be referred to as markers, trace tags, anti-counterfeiting markers and/or taggants and for the purposes of this disclosure will be collectively referred to as “Marker” or “Markers”). Additionally, eliminating the presence of water can substantially increase DNA stability thus avoiding the possibility of hydrolysis and metal-assisted nucleic acid degradation characteristically associated with aqueous systems.

In one embodiment, the present invention provides a method of marking a hydrophobic medium with a nucleic acid marker, the method includes: providing a trialkylammonium salt of the nucleic acid marker, a tetraalkylphosphonium salt of the nucleic acid marker or a tetraarylphosphonium salt of the nucleic acid marker; and incorporating the trialkylammonium salt of the nucleic acid marker, the tetraalkylphosphonium salt of the nucleic acid marker or the tetraarylphosphonium salt of the nucleic acid marker into the hydrophobic medium.

In another embodiment, the present invention provides a method of authenticating a hydrophobic medium with a nucleic acid marker, the method includes providing a trialkylammonium salt, a tetraalkylphosphonium salt or a tetraarylphosphonium salt of the nucleic acid marker; and incorporating the trialkylammonium salt, the tetraalkylphosphonium salt or the tetraarylphosphonium salt of the nucleic acid marker into the hydrophobic medium; and detecting the nucleic acid marker and thereby authenticating the hydrophobic medium.

In still another embodiment, the present invention provides a method of authenticating and tracking a hydrophobic medium with a nucleic acid marker, the method includes: providing a trialkylammonium salt of the nucleic acid marker, a tetraalkylphosphonium salt of the nucleic acid marker or a tetraarylphosphonium salt of the nucleic acid marker; incorporating the trialkylammonium salt of the nucleic acid marker, the tetraalkylphosphonium salt of the nucleic acid marker or the tetraarylphosphonium salt of the nucleic acid marker into the hydrophobic medium; introducing the trialkylammonium salt of the nucleic acid marker, the tetraalkylphosphonium salt of the nucleic acid marker or the tetraarylphosphonium salt of the nucleic acid marker into the stream of commerce; providing a sample of the hydrophobic medium from the stream of commerce; and detecting the nucleic acid marker in the sample of the hydrophobic medium and thereby authenticating the hydrophobic medium.

In one embodiment, the present invention provides a method of preparation of hydrophobic and organic solvent soluble DNA Markers. The hydrophobic DNA can be prepared by converting the water soluble DNA form, such as a sodium salt, into a trialkylammonium salt in two steps, as depicted in the Scheme 1 below.

In one embodiment, the first step of the method involves the synthesis of a trialkylammonium salt R₁R₂R₃NH⁺X⁻. This step is accomplished by treating amine R₁R₂R₃N with acid HX in an appropriate solvent. Alternatively, a solvent-free process can be used. The choice of trialkylammonium salt, and more specifically, R₁, R₂, R₃ and X can be modified according to desired solubility requirements and may be tailored to an intended use or application. In the event that the desired trialkylammonium salt R₁R₂R₃NH⁺X⁻ salt is commercially available, this synthetic step may be skipped and the commercially available trialkylammonium salt may be used directly in the second step described below.

The second step involves a salt exchange reaction between water soluble DNA, such as a sodium salt, and a molar excess of the trialkylammonium salt R₁R₂R₃NH⁺X⁻, provided in Step 1. The resulting product is a hydrophobic DNA, which may be purified and isolated by any desalting methods known in the art, including but not limited to, diafiltration, dialysis or size-exclusion chromatography. It will be recognized by those skilled in the art that in simplest case wherein one or two of the R groups are hydrogen, the hydrophobic DNA can be monoalkylammonium or dialkylammonium salts, respectively.

Alternatively, in another embodiment, the hydrophobic DNA that is soluble in organic solvents can be prepared by a salt exchange reaction between water soluble DNA, such as a sodium salt, and a molar excess of a tetraalkylphosphonium or a tetraarylphosphonium salt. R₁R₂R₃R₄P⁺X⁻, as depicted in Scheme 2. The resulting product is hydrophobic DNA, which may be purified and isolated by any of the desalting methods known in the art, including, but not limited to diafiltration, dialysis or size-exclusion chromatography.

### DETAILED DESCRIPTION

### Definitions

DNA is a deoxyribonucleic acid, which is a biopolymer composed of simpler nucleotide units. Each nucleotide includes a nitrogen-containing base—either guanine (G), adenine (A), thymine (T), or cytosine (C), a monosaccharide sugar, deoxyribose, and a phosphate group. The nucleotides are bonded to one another in a chain by covalent bonds between the five member pentose sugar moiety at the 3′ and 5′ pentose ring positions of one nucleotide and the phosphate group of the next, resulting in an alternating sugar-phosphate backbone. A fairly short chain of nucleic acids are referred to as oligonucleotides, and usually have a length of approximately 15 to 100 nucleotides. Nucleic acids useful in the practice of the present invention may have any length from about five to about 10,000 nucleotides.

A DNA Marker, useful in the practice of the present invention, can he single-stranded (ss), where the nucleotides form a single, straight polymeric chain, or double-stranded (ds), where two anti-parallel chains form a double helical structure. Double stranded DNA structures are generally described as composed of base pairs (bp) of complementary nucleotides (A-T and C-G base pairs) interacting through hydrogen bonds.

In one embodiment of the present invention, a DNA Marker is an oligodeoxynucleotide—a single stranded DNA of about 15 to about 100 nucleotides. In another embodiment, the DNA Marker is a single stranded DNA of about 10 to about 10,000 nucleotides in length. In another embodiment, the DNA Marker is a double stranded DNA molecule of about 15 to about 1,000 base pairs. In another embodiment the DNA Marker is a double stranded DNA of about 10 to about 10,000 base pairs in length.

A schematic description of the above DNA forms is depicted below (Scheme 3).
ss [symbol] N₁-N₂-N₃ . . . Nₙ

[symbol]

Wherein ss refers to single-stranded DNA and N₁, N₂, N₃ . . . Nₙ denote nucleotides as defined above.
ds [symbol] BP₁-BP₂-BP₃ . . . BPₙ

[symbol]

Wherein ds refers to double-stranded DNA and BP₁, BP₂, BP₃ . . . BPₙ denote nucleotide base pairs as defined above,
oligo [symbol] N₁-N₂-N₃ . . . BPₙ

[symbol]

Wherein oligo refers to oligonucleotide and N₁, N₂, N₃ . . . Nₙ denote nucleotides as defined above.

Scheme 3: Schematic Representation of DNA Structures

Alkyl, as used herein, refers to a saturated branched or straight chain monovalent hydrocarbon radical of a specified number of carbon atoms. Thus, the term alkyl includes, but is not limited to, methyl (C₁ alkyl), ethyl (C₂ alkyl), propyl and isopropyl (C₃ alkyl), n-butyl, isobutyl, sec-butyl and t-butyl (C₄ alkyl).

Alkenyl refers to branched or straight chain hydrocarbon radical having at least one double bond between two carbon atoms.

Alkynyl refers to branched or straight chain hydrocarbon radical having at least one triple bond between two carbon atoms.

Cycloalkyl as used herein means a saturated monocyclic, polycyclic or bridged hydrocarbon ring system substituent or linking group. In a substituted cycloalkyl ring, the substituent is bonded to a ring carbon atom replacing a hydrogen atom. For example, a substituted C₃-C₁₀ cycloalkyl designates a ring of three to ten carbon atoms with one or more substituents replacing one or more hydrogen atoms.

Heterocyclyl as used herein means a saturated, partially unsaturated or unsaturated monocyclic, polycyclic or bridged hydrocarbon ring system substituent or linking group, wherein at least one ring carbon atom has been replaced with a heteroatom such as, but not limited to nitrogen, oxygen, sulfur, alternatively, a ring carbon atom of the heterocyclyl moiety can be replaced with a selenium, boron or phosphorus atom. A heterocyclyl ring system can be a ring system having one, two, three or four nitrogen ring atoms, or a ring system having zero, one, two or three nitrogen ring atoms and one oxygen or sulfur ring atom. The heterocyclic ring system can include more than one ring heteroatom. A heterocyclyl substituent is derived by the removal of one hydrogen atom from a single carbon or nitrogen ring atom. Heterocyclyl includes, but is not limited to, furyl, thienyl, 2H-pyrrole, 2-pyrrolinyl, 3-pyrrolinyl, pyrrolidinyl, pyrrolyl, 1,3-dioxolanyl, oxazolyl, thiazolyl, imidazolyl, 2-imidazolinyl, imidazolidinyl, 2-pyrazolinyl, pyrazolidinyl, pyrazolyl, isoxazolyl, isothiazolyl, oxadiazolyl, triazolyl, thiadiazolyl, tetrazolyl, 2H-pyranyl, 4H-pyranyl, pyridinyl, piperidinyl, 1,4-dioxanyl, morpholinyl, 1,4-dithianyl, thiomorpholinyl, pyridazinyl, pyrimidinyl, pyrazinyl, piperazinyl, azepanyl, diazepinyl, indolizinyl, indolyl, isoindolyl, 3H-indolyl, indolinyl, benzo[b]furyl, benzo[b]thienyl, 1H-indazolyl, benzimidazolyl, benzothiazolyl, purinyl, 4H-quinolizinyl, quinolinyl, isoquinolinyl, cinnolinyl, phthalzinyl, quinazolinyl, quinoxalinyl, 1,8-napthyridinyl, pteridinyl, quinuclidinyl.

As noted above, heterocyclyl also includes aromatic heterocycles, such as pyrrolyl, pyrazolyl, imidazolyl, triazolyl, oxazolyl, isoxazolyl, thiazolyl, isothiazolyl, furyl, thienyl, pyridyl, pyrazinyl, pyrimidinyl, and can be optionally substituted, for instance with alkyl. Heterocyclyl also includes bicyclic heterocyclyls with one or both rings having a heteroatom, e.g. imidazopyrazinyl, benzofuranyl, benzodioxolyl, benzothiophenyl, and quinolinyl.

Arylalkyl means an optionally substituted aryl group attached to the end carbon atom of an alkyl group such as, for instance C₁-C₄ alkyl.

Aryl means an aromatic, unsaturated a-electron conjugated monocyclic or polycyclic hydrocarbon ring system substituent or linking group of six, eight, ten or fourteen carbon atoms. An aryl group is derived by the removal of one pair of hydrogen atoms from neighboring carbon ring atoms. Aryl includes, but is not limited to, phenyl, naphthalenyl, azulenyl and anthracenyl.

Compounds useful in the practice of the present invention as hydrophobic DNA Markers include chemical species represented schematically by the simplified structures shown in the scheme below (Scheme 4).
ss [symbol] N₁-N₂-N₃ . . . Nₙ [symbol] R₁R₂R₃NH Salt

Wherein ss refers to single-stranded DNA and N₁, N₂, N₃ . . . Nₙ denote nucleotides as defined above, and R₁, R₂, and R₃ are defined below,
ds [symbol] BP₁-BP₂-BP₃ . . . BPₙ [symbol] R₁R₂R₃NH Salt

Wherein ds refers to double-stranded DNA and BP₁, BP₂, BP₃ . . . BPₙ denote nucleotide base pairs as defined above, and R₁, R₂, and R₃ are defined below,
oligo [symbol] N₁-N₂-N₃ . . . Nₙ [symbol] R₁R₂R₃NH Salt

Wherein oligo refers to oligonucleotide and N₁, N₂, N₃ . . . Nₙ denote nucleotides as defined above, and R₁, R₂, and R₃ are defined below.

Scheme 4: Schematic Representation of Hydrophobic DNA Structures Based on Ammonium Salts (Ionic Charges Omitted for Simplicity)

In one embodiment, the marker hydrophobic DNA includes a phosphonium salt-based species represented schematically by the simplified structures shown in the scheme below (Scheme 5).
ss [symbol] N₁-N₂-N₃ . . . Nₙ [symbol] R₁R₂R₃R₄P Salt

Wherein ss refers to single-stranded DNA and N₁, N₂, N₃ . . . Nₙ denote nucleotides as defined above, and R₁, R₂, and R₃ are defined below,
ds [symbol] BP₁-BP₂-BP₃ . . . BPₙ [symbol] R₁R₂R₃R₄P Salt

Wherein ds refers to double-stranded DNA and BP₁, BP₂, BP₃ . . . BPₙ denote nucleotide base pairs as defined above, and R₁, R₂, and R₃ are defined below,
oligo [symbol] N₁-N₂-N₃ . . . Nₙ [symbol] R₁R₂R₃R₄P Salt

Wherein oligo refers to oligonucleotide and N₁, N₂, N₃ . . . Nₙ denote nucleotides as defined above, and R₁, R₂, and R₃ are defined below.

Scheme 5: Schematic Representation of Hydrophobic DNA Structures Based on Phosphonium Salts (Ionic Charges Omitted for Simplicity)

R₁, R₂, R₃, and R₄, in the schemes shown above may each be independently selected from hydrogen, C₁-C₂₂ alkyl, C₃-C₈ cycloalkyl, alkenyl, C₂-C₂₂ alkynyl, heterocyclyl, aryl, and arylalkyl each optionally substituted with one or more oxygen, nitrogen, sulfur or functional groups such as hydroxyl, carboxyl, amino, cyano, alkyl, alkenyl, alkynyl or azido.

Alternatively, a pair of R groups independently selected from R₁, R₂, R₃, and R₄, can form a ring between one another.

In another embodiment, a hydrophobic DNA Marker useful in the practice of the present invention is prepared in a two step process, wherein the precursor trialkylammonium salt is synthesized first, according to the Scheme 6.

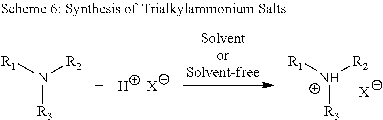

The choice of trialkylammonium salt, and more specifically, R₁, R₂, R₃ groups may be determined according to desired solubility requirements of the DNA usages and can be tailored to an intended application. This step may be accomplished by treating amine R₁R₂R₃N with acid. HX in an appropriate solvent. Alternatively, solvent-free process can be used. It will be recognized by those skilled in the art that in simplest case of R groups being reduced to hydrogen, the hydrophobic DNA described will assume the form of mono- or dialkylammonium salts.

The choice of counterion X⁻ may be determined according to desired solubility requirements and accessibility of the acid used, HX. A suitable counterion can be for instance, but is not limited to: fluoride, chloride, bromide, iodide, sulfate, nitrate, orthophosphate, pyrophosphate, tosylate, mesylate, acetate, benzoate, salicylate or perchlorate.

In the case of hydrophobic DNA Markers based on tetraalkylphosphonium or tetraarylphosphonium counterions, the required precursor phosphonium salt may be synthesized from the appropriate phosphine. Alternatively, some of the commercially available phosphonium salts can be used instead. Here, the choice of R₁, R₂, R₃ and R₄ groups may also he determined according to desired solubility requirements of the DNA and is tailored to an intended use or application.

The second step of hydrophobic DNA Marker preparation involves a salt exchange reaction between water soluble DNA, such as lithium, sodium or potassium salt, and molar excess of trialkylammonium salt R₁R₂R₃NH⁺X⁻, tetraalkylphosphonium or tetrarylphosphonium salt R₁R₂R₃R₄P⁺X⁻, synthesized in Step 1 (Scheme 7). The resulting product, hydrophobic DNA, may be purified and isolated by any desalting methods known in the art, such as diafiltration, dialysis or size-exclusion chromatography.

The above reactions may also employ combinations of ammonium and phosphonium salts.

In addition, Markers may be formed of tetraalkylammonium salts, where R₁ through R₄ as well as X are defined as above. The Markers of Scheme 8 may be employed as security or anti-counterfeiting tags.

The hydrophobic nucleic acid salt Markers of the present invention are suitable for addition to any hydrophobic medium. For instance, hydrophobic nucleic acid salt Markers of the present invention can be used to provide authentication and tracking of a hydrophobic material, whether liquid, solid or gel. Hydrophobic materials, such as crude oils, petroleums, petroleum-based or petroleum-derived materials such as polymers, plastics, building materials, foods, medicines, or cosmetics, motor, engine and/or jet fuels, kerosene, diesel fuel, oils, grease or gels. The hydrophobic materials may be used in any industry, including excipients, medicines, foods, industrial applications and in construction.

In one embodiment, the hydrophobic nucleic acid salt Markers of the present invention can be labelled with a detectable moiety, e.g. an optical marker, such as a fluorescent molecule, which may be covalently bonded to the nucleic acid.

In another embodiment,the hydrophobic nucleic acid salt Markers of the present invention can be incorporated into hydrophobic coatings useful for identifying or tagging solid objects. Suitable coatings include paints, varnishes, lacquers and inks.

The hydrophobic nucleic acid salt Markers of the present invention can be recovered from the carrier hydrophobic medium by eluting with an aqueous salt solution, such as for instance, sodium chloride, permitting ion exchange between the aqueous and hydrophobic phases and detection of the nucleic acid directly e.g. optically by detection of a bound marker dye or fluorophore, or by hybridization directly using a probe having a sequence complementary to the nucleic acid Marker; or hybridization after amplification by PCR, isothermal amplification or other standard methods well known in the art. Alternatively, nucleic acid Marker may be captured by binding with a hybridization probe and its nucleic acid sequence determined by any of the currently available nucleic acid sequencing methods to confirm its authenticity.

Dilution or adulteration of the hydrophobic medium containing the nucleic acid Marker can be detected after shipping or recovery from the stream of commerce by quantifying the amount of nucleic acid Marker remaining per unit volume in a sample of the hydrophobic medium and comparing with the amount of nucleic acid Marker remaining per unit volume of the hydrophobic medium present in a sample of the hydrophobic medium obtained prior to shipping or entry into the stream of commerce.

### EXAMPLES

### Example 1

### Synthesis of Tributylammonium Chloride

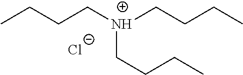

To neat tributylamine (10 mmole) was slowly added 1 M aqueous hydrochloric acid (11 mmole) and the mixture was stirred at room temperature for 1 hour. The reaction mixture was concentrated in vacuo to give a thick, viscous product which crystalized upon standing.

### Example 2

### Synthesis of Trihexylammonium Chloride

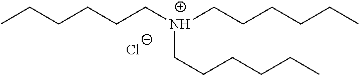 [SMILES: CCCCCC[NH+](CCCCCC)CCCCCC ; [Cl-]]

To neat trihexylamine (10 mmole) was slowly added concentrated aqueous hydrochloric acid (11 mmole) and the mixture was stirred at room temperature for 1 hour. The reaction mixture was concentrated in vacuo to give a thick, viscous product.

### Example 3

### Synthesis of Trioctylammonium Chloride

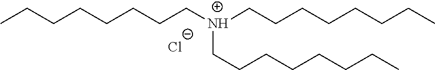 [SMILES: CCCCCCCC[NH+](CCCCCCCC)CCCCCCCC ; [Cl-]]

To trioctylamine (10 mmole) dissolved in ethanol, was slowly added concentrated aqueous hydrochloric acid (11 mmole) and the mixture was stirred at room temperature for 1 hour. The reaction mixture was concentrated in vacuo to give a thick, viscous product.

### Example 4

### Preparation of DNA Trialkylammonium Salt

Aqueous solution of DNA sodium salt is treated with a 10-100 fold molar access of trialkylammonium chloride salt. The mixture is incubated at room temperature for 20 min to 1 hour and purified by diafiltration.

### Example 5

### Preparation of DNA Tetraalkylphosphonium Salt

An aqueous solution of DNA sodium salt is treated with a 10-100 fold molar access of tetraalkylphosphonium salt. The mixture is then incubated at room temperature for 20 min to 1 hour and purified by diafiltration.

What is claimed is:

1. A method of marking a hydrophobic medium with a nucleic acid marker, the method comprising:
providing a trialkylammonium salt of the nucleic acid marker, a tetraalkylphosphonium salt of the nucleic acid marker or a tetraarylphosphonium salt of the nucleic acid marker; and
incorporating the trialkylammonium salt of the nucleic acid marker, the tetraalkylphosphonium salt of the nucleic acid marker or the tetraarylphosphonium salt of the nucleic acid marker into the hydrophobic medium.

2. The method of claim 1, wherein the sequence of the nucleic acid marker encodes information specific to the hydrophobic medium.

3. The method of claim 1, wherein the nucleic acid marker comprises DNA.

4. The method of claim 3, wherein the DNA comprises a single stranded molecule of from about 10 to about 10,000 bases.

5. The method of claim 4, wherein the DNA is a single stranded molecule of from about 15 to about 1,000 bases.

6. The method of claim 3, wherein the DNA is a double stranded molecule of from about 10 to about 10,000 base pairs.

7. The method of claim 6, wherein the DNA is a double stranded molecule of from about 15 to about 1,000 base pairs.

8. The method of claim 1, wherein the nucleic acid marker is a non-natural sequence.

9. A method of authenticating a hydrophobic medium with a nucleic acid marker, the method comprising:
providing a trialkylammonium salt of the nucleic acid marker, a tetraalkylphosphonium salt of the nucleic acid marker or a tetraarylphosphonium salt of the nucleic acid marker; and
incorporating the trialkylammonium salt of the nucleic acid marker, the tetraalkylphosphonium salt of the nucleic acid marker or the tetraarylphosphonium salt of the nucleic acid marker into the hydrophobic medium; and
detecting the nucleic acid marker and thereby authenticating the hydrophobic medium.

10. The method of claim 9, wherein the sequence of the nucleic acid marker encodes information specific to the hydrophobic medium.

11. The method of claim 9, wherein the nucleic acid marker comprises DNA.

12. The method of claim 11, wherein the DNA is a single stranded molecule of from about 10 to about 10,000 bases.

13. The method of claim 12, wherein the DNA is a single stranded molecule of from about 15 to about 1000 bases.

14. The method of claim 11, wherein the DNA is a double stranded molecule of from about 10 to about 10,000 base pairs.

15. The method of claim 14, wherein the DNA is a double stranded molecule of from about 15 to about 1000 base pairs.

16. The method of claim 9, wherein the nucleic acid marker is a non-natural sequence.

17. The method of claim 9, wherein the hydrophobic medium is a petroleum-derived material.

18. The method of claim 9, wherein the hydrophobic medium is a petroleum-derived material is an oil, a crude oil, a fuel, a diesel fuel, a polymer, a building material, an asphalt, a medicine, a cosmetic, or a plastic.

19. The method of claim 9, wherein the hydrophobic medium is an oil comprising food material, medicinal material or cosmetic material.

20. The method of claim 9, wherein the hydrophobic medium is a gel comprising food material, medicinal material or cosmetic material.

21. A method of authenticating and tracking a hydrophobic medium with a nucleic acid marker, the method comprising:
providing a trialkylammonium salt of the nucleic acid marker, a tetraalkylphosphonium salt of the nucleic acid marker or a tetraarylphosphonium salt of the nucleic acid marker;
incorporating the trialkylammonium salt of the nucleic acid marker, the tetraalkylphosphonium salt of the nucleic acid marker or the tetraarylphosphonium salt of the nucleic acid marker into the hydrophobic medium;
introducing the trialkylammonium salt of the nucleic acid marker, the tetraalkylphosphonium salt of the nucleic acid marker or the tetraarylphosphonium salt of the nucleic acid marker into the stream of commerce;
providing a sample of the hydrophobic medium from the stream of commerce; and
detecting the nucleic acid marker in the sample of the hydrophobic medium and thereby authenticating the hydrophobic medium.
